(19) United States
(12) Patent Application Publication
(10) Pub. No.: US 2002/0004497 A1
(43) Pub. Date: Jan. 10, 2002

(54) Combination injection preparation

(76) Inventors: Jorma Tolonen, Espoo (FI); Antti Malmivaara, Helsinki (FI)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/901,867

(22) Filed: Jul. 9, 2001

Related U.S. Application Data
(63) Continuation of application No. 09/359,238, filed on Jul. 22, 1999.

(30) Foreign Application Priority Data
Jan. 10, 1997 (FI) PCT/FI97/00008
Jan. 10, 1996 (FI) FI 960121

(51) Int. Cl. A61K 31/56

(52) U.S. Cl. 514/169

(57) ABSTRACT

The invention belonging to the field of clinical pharmacology is based on a combination of a glucocorticoid and an antiinflammatory analgesic to be administered as a targeted drug in the form of an injection. The preparation exhibits a surprisingly rapid, effective and long-acting effect on musculoskeletal soft tissue disorders without side effects.

The object of the present invention is a new injection preparation comprising, in combination, a glucocorticoid and a NSAID antiinflammatory analgesic (non-steroidal antiinflammatory drug) as well as the use of these active agents for the preparation of such an injection preparation to be injected intralesionally directly into the site of pain or inflammation, for the treatment of pain and inflammatory conditions associated with musculoskeletal soft tissue disorders.

Numerous medical treatment methods have been tested for the treatment of musculoskeletal soft tissue disorders, such as cortisone injections administered to the site of pain, and orally administered antiinflammatory analgesics. They are used in our country and elsewhere relatively widely without reliable scientific evidence. Cortisone injections have, however, their well-known adverse effects and often repeated injections are not recommended. Nearly all orally administered antiinflammatory analgesics have side effects on the gastro-intestinal tract. As they, in addition, are distributed into the interstitial fluid of the whole body, the drug concentrations in the tissue after oral administration remain relatively small, and the benefit reached by them is often questionable.

As the orally administered antiinflammatory analgesics have the aforementioned adverse effects and deficiencies, there are injection formulations of these drugs which have been administered parenterally intramuscularly to a patient, not, however, directly into the site of inflammation and pain. Also when using intramuscularly administered antiinflammatory analgesics, the drug concentrations in the tissue remain relatively small.

According to the invention it has now surprisingly been discovered that very good treatment results are reached in the treatment of musculoskeletal soft tissue disorders, such as epicondylitis (usually tennis elbow) by injecting an antiinflammatory analgesic and a glucocorticoid as a combination preparation directly into the site of pain. By means of injectable antiinflammatory analgesics administered intralesionally into the site of pain, high concentrations of the drug are reached in the tissue, which are even a thousand times higher as compared to a treatment with orally or intramuscularly or intravenously administered drug. Although the effect of a drug usually increases in relation to the logarithmic increase of the drug concentration or dosage, the thousendfold levels reached by the intralesional injection treatment also multiply the local effect without increasing the side effects of the drugs.

The object of the invention is thus an injection preparation which contains

a glucocorticoid

a non-steroidal antiinflammatory analgesic (NSAID)

a carrier suitable for injection purposes, and optionally other pharmacologically acceptable adjuvants and/or additives.

The object of the invention is also the use of a non-steroidal antiinflammatory analgesic in combination with a glucocorticoid for the preparation of an injection preparation to be injected intralesionally directly into the site of pain, for the treatment of pain and inflammatory conditions associated with musculoskeletal soft tissue disorders. By combining a non-steroidal antiinflammatory analgesic and a glucocorticoid, drugs having different mechanisms of action can be combined in order to reach the most optimal effect of relief of inflammation and pain. It is also possible to use a combination of two or more antiinflammatory analgesics or a combination of two or more glucocorticoid compounds.

The antiinflammatory analgesic to be used according to the invention is preferably selected from the following groups:

phenyl acetic acid or phenyl propionic acid derivatives such as ketoprofen, ibuprofen, naproxen, alclofenac, diclofenac, fenoprofen, tolmetin, suprofen, ketorolac, pyrazolone derivatives, such as phenylbutazone, oxyphenbutazone, metamizol, salicylic acid derivatives, such as salicylic acid, salicylic acid amide, acetylsalicylic acid, indole derivatives, such as indomethacin, sulindac, anthranilic acids and its analogues, such as flufenamic acid, mefenamic acid, niflumic acid, tolfenamic acid, oxicams, such as piroxicam, tenoxicam, lefetamine, nabumetone.

According to one embodiment of the invention the antiinflammatory analgesic is ketoprofen, diclofenac, or indomethacin.

The glucocorticoid used in the invention is selected e.g. from the following group of compounds: cortisone, hydrocortisone, prednisone, prednisolone, methylprednisolone, triamcinolone, betamethasone, dexamethasone.

According to the invention, the active agents are injected into the site of pain of a patient in an amount sufficient to achieve the desired treatment result. This amount depends, of course, on both the condition of the patient and the pharmaceutical agents to be used, and this amount can be determined by a person skilled in the art. The amount of antiinflammatory analgesic typically used is in the range of 1 to 1000 mg/single injection dose. As an example it can be mentioned that when using ketoprofen, the dosage used varies from 10 to 100 mg/single injection dose, and from 1 to 100 mg/single injection dose when using diclofenac.

The amount of the corticoid type compound also depends both on the condition of the patient and the drug to be used, and it can also be determined by a person skilled in the art. The amount can thus vary in the range of 1 to 100 mg/single injection dose, depending on the drug.

The injection frequence to be used according to the invention depends naturally on the condition of the patient and the dosage level used. We have, as appears later, treated severe epicondylitis and other musculoskeletal soft tissue disorders by injecting two single injections at an interval of a week, and thereby reached good results.

The object of the invention is also an injection preparation which contains a corticoid in combination with a non-steroidal antiinflammatory analgesic in a carrier suitable for injection, whereby suitable adjuvants and/or additives are added, when necessary or desired.

The amount of the active agents in the injection preparation according to the invention can vary within relatively large ranges depending on the drug and the other additives. An appropriate amount of each of the active agents is generally in the range of 0.1 to 5% by weight, depending on the drug and the formulation.

The carrier is preferably sterile water or a physiological (0.9%) sodium chloride solution suitable for injection. Also other pharmaceutically acceptable organic solvents suitable for injection purposes, and mixtures thereof with water, may come into question.

In the preparation according to the invention adjuvants and additives known per se can be used. Such agents are e.g. preservative agents, buffers and other agents suitable for adjusting the pH-value, agents for adjusting the osmotic pressure, viscosity adjustment agents, solubility improving agents, stabilizing agents, surface-active agents and anesthetics. The selection of the type and the amount of carrier and other adjuvants and additives can be done by a person skilled in the art.

As suitable preservative agents benzalkonium chloride, benzyl alcohol, thiomersal, chlorhexidine and comparable agents, or mixtures thereof, can be mentioned.

As suitable buffers e.g. phosphate buffers, citrate buffers, and borate buffers come into question. For adjusting the pH-value, conventional pH adjustment agents, such as inorganic and organic bases and acids, can be used. As bases e.g. sodium hydroxide, amines, e.g. alcanol amines or amino acids, e.g. arginine or lysine, come into question. As an acid, e.g. hydrochloric acid is useful. For adjusting the osmotic pressure, agents well-known for this purpose, such as sodium chloride, glycerol, mannitol, sorbitol, lactose, sodium borate or corresponding agents, can be used.

When desired, also viscosity adjustment agents, typically various cellulose derivatives, such as sodium carboxymethyl cellulose, can be used in the preparation. In some cases it can be preferable to add solubility enhancing agents or stabilizing agents, e.g. polyvinylpyrrolidone of which various qualities are commercially available e.g. under the name Kollidon.

According to one advantageous embodiment of the invention an injection solution ready for use is prepared from a so-called pre-preparation or pre-pack, “kit”. The pre-pack can contain e.g. two separate components, i.e. a component containing a glucocorticoid, and a component containing an antiinflammatory analgesic, as well as means for combining the active agents to form a preparation designed to be injected, which preparation contains a pharmacologically acceptable carrier suitable for injecting and optionally further additives and adjuvants. Preferably, both the component containing the glucocorticoid and the component containing the antiinflammatory analgesic are in injectable form, each of them containing a pharmacologically acceptable carrier suitable for injection purposes, and optionally further adjuvants and/or additives. The components can advantageously be joined in a triple ampule, which additionally contains a suitable anesthetic for preventing possible smarting, such as lidocaine or bupivacaine, in combination with adrenaline when necessary, if there are no contraindications for the use of adrenaline. The anesthetic is given prior to the administration of the actual drugs. At the moment of use, the active agents of the components can be joined in an appropriate manner, e.g. by joining the contents of the ampules, e.g. by drawing the injection solution contained in each of the ampules into the same syringe, wherein they are mixed together. By this arrangement, any preservation problems caused by the possible incompatibility of the drugs are avoided.

The following examples illustrate the invention.

### EXAMPLE 1

The following injection solution compositions 1 and 2 are prepared separately.

| | | |
|---|---|---|
| ketoprofen | 5 | mg |
| arginine | 36 | mg |
| benzyl alcohol | 25 | mg |
| citric acid monohydrate q.s. ad pH 6.5 | 1 | ml |
| water, aq. ad. inj. ad | | |

| | | |
|---|---|---|
| betamethasone acetate | 3 | mg |
| betamethasone disodium phosphate | 3 | mg |
| (respond betamethasone) | | |
| disodium phosphate anhydride | 7.1 | mg |
| sodium dihydrogen phosphate monohydrate | 3.4 | mg |
| sodium edetate | 0.1 | mg |
| benzalkonium chloride | 0.2 | mg |
| water, aq. ad. inj. ad | 1 | ml |

Two ml of the solution having the above composition 1 is used as the injection solution of the antiinflammatory analgesic and one ml of the solution having the above composition 2 is used as the corticoid solution. These solutions are mixed together by drawing them in succession into a syringe prior to injecting.

### EXAMPLE 2

Analogously to Example 1, an injection solution is prepared from the injection solutions having the following compositions 1 and 2.

| | | |
|---|---|---|
| ketoprofen | 50 | mg |
| arginine | 36 | mg |
| benzyl alcohol | 25 | mg |
| citric acid monohydrate q.s. ad pH 6.5 | 1 | ml |
| water, aq. ad. inj. ad. | | |

| | | |
|---|---|---|
| methylprednisolone acetate | 40 | mg |
| macrogol 4000 | 29 | mg |
| sodium chloride | 8.7 | mg |
| myristyl-γ-picoline chloride | 0.19 | mg |
| water, aq. ad. inj. ad. | 1 | ml |

### EXAMPLE 3

Analogously to Example 1, an injection solution is prepared from the injection solutions having the following compositions 1 and 2.

| | | |
|---|---|---|
| ketoprofen | 50 | mg |
| arginine | 36 | mg |
| benzyl alcohol | 25 | mg |
| citric acid monohydrate q.s. ad pH 6.5 | 1 | ml |
| water, aq. ad. inj. ad | | |

| | | |
|---|---|---|
| methylprednisolone acetate | 40 | mg |
| macrogol 3000 | 30 | mg |
| sodium chloride | 5 | mg |
| Kollidon PF 12 | 14 | mg |
| Polysorb 80 | 2 | mg |
| benzyl alcohol | 10 | mg |
| water, aq. ad. inj. ad | 1 | ml |

### EXAMPLE 4

Analogously to Example 1, an injection solution is preparedd from the injection solutions having the following compositions 1 and 2.

| | | |
|---|---|---|
| ketoprofen | 50 | mg |
| arginine | 36 | mg |
| benzyl alcohol | 25 | mg |
| citric acid monohydrate q.s. ad pH 6.5 | 1 | ml |
| water, aq. ad. inj. ad | | |

| | | |
|---|---|---|
| methylprednisolone sodium succinate | 40 | mg |
| respond methylprednisolone | | |
| lactose, anh. | 25 | mg |
| sodium dihydrogen phosphate monohydrate | 1.8 | mg |
| sodium phosph. sicc. | 17.5 | mg |
| benzyl alcohol | 9 | mg |
| water, aq. ad. inj. ad | 1 | ml |

### EXAMPLE 5

Analogously to Example 1, an injection solution is prepared from the injection solutions having the following compositions 1 and 2.

| | | |
|---|---|---|
| ketoprofen | 50 | mg |
| arginine | 36 | mg |
| benzyl alcohol | 25 | mg |
| citric acid monohydrate q.s.ad pH 6.5 | 1 | ml |
| water, aq. ad. inj. ad | | |

| | | |
|---|---|---|
| triamcinolone acetonide | 50 | mg |
| sodium carboxymethylcellulose | 7.5 | mg |
| sodium chloride | 6.6 | mg |
| Polysorb 80 | 0.4 | mg |
| benzyl alcohol | 15 | mg |
| water, aq. ad. inj. ad | 1 | ml |

### EXAMPLE 6

Analogously to Example 1, an injection solution is prepared from the injection solutions having the following compositions 1 and 2.

| | |
|---|---|
| ketoprofen | 50 mg |
| arginine | 36 mg |
| benzyl alcohol | 25 mg |
| citric acid monohydrate q.s. ad pH 6.5 | 1 ml |
| water, aq. ad. inj. ad | |

| | |
|---|---|
| triamcinolone hexacetonide | 20 mg |
| benzyl alcohol | 9 mg |
| Polysorb 80 | 4 mg |
| sorbitol | 450 mg |
| sodium hydroxide/hydrochlorid acid ad pH 5.8/6.5 | 1 ml |
| water, aq. ad. inj. ad | |

### EXAMPLE 7

Analogously to Example 1, an injection solution is prepared from the injection solutions having the following compositions 1 and 2.

| | |
|---|---|
| diclofenac sodium | 25 mg |
| mannitol | 6 mg |
| sodium pyrosulfis. | 0.67 mg |
| benzyl alcohol | 40 mg |
| propylene glycol | 200 mg |
| sodium hydroxide ad pH 8.0 | 1 ml |
| water, aq. ad. inj. ad | |

| | |
|---|---|
| methylprednisolone acetate | 40 mg |
| macrogol 4000 | 20 mg |
| sodium chloride | 8.7 mg |
| myristyl-γ-picoline chloride | 0.19 mg |
| water, aq. inj. ad | 1 ml |

As the injection solution of the antiinflammatory analgesic, 3 ml of the solution having the above composition 1 is used, and as the corticoid solution, 1 ml of the solution having the above composition 2 is used. These solutions are mixed together by drawing them in succession into a syringe prior to injecting.

### EXAMPLE 8

Analogously to Example 1, an injection solution is prepared by combining the following compositions 1 and 2.

| | |
|---|---|
| indomethacin sodium | 50 mg |
| respond indomethacin | |
| monosodium phosphate | 27.1 mg |
| sodium hydroxide | 12 mg |
| water, aq. ad. inj. ad | 10 ml |

| | |
|---|---|
| methylprednisolone acetate | 40 mg |
| macrogol 3000 | 30 mg |
| sodium chloride | 5 mg |
| Kollidon PF 12 | 14 mg |
| polysorbate 80 | 2 mg |
| benzyl alcohol | 10 mg |
| water, aq. ad. inj. ad | 1 ml |

As the injection solution of the antiinflammatory analgesic, 3 ml of the solution having the above composition 1 is used, and as the corticoid solution, 1 ml of the solution having the above composition 2 is used. These compositions are mixed together by drawing them in succession into a syringe prior to injecting.

Combination preparations can be prepared by analogously using other, above mentioned NSAIDs.

### Test Report

We have treated patients by administering combination injections of a glucocorticoid compound and an antiinflammatory analgesic as “a targeted means” directly into the site of pain. There are no published studies of such an alternative for the treatment of epicondylitis or a corresponding disease, nor is such a treatment disclosed in any textbook in the art. We have used an anesthetic-cortisone-ketoprofen combination preparation in the treatment of epicondylitis and comparable conditions of musculoskeletal soft tissue disorders. We have treated various musculoskeletal disorders with the combination preparation, i.a. rotator cuff tendinitis of the shoulder joint, bursitis, peritendinitis, tendovaginitis and insertion tendinitis, post-traumatic soft tissue disorders and distended joints. Other indications are pain conditions in the neck and the back as well as joint disorders. Among the diseases treated there have been sixteen patients with chronic tennis elbow, whose disease had lasted from two months to one year. All treated patients recovered completely or nearly completely after two injections of the combination (given at one-week intervals) within two to three weeks. The effect has lasted at least for three months from the first injection given. The effect has been very surprising, as chronic tennis elbow, for the treatment of which we mainly used the preparation, has generally been considered as a condition, which is hardly cured by drug treatment. As an anesthetic we have used bupivacaine administered prior to the combination preparation; in this case, the antiinflammatory analgesic used, ketoprofen, causes hardly any smarting. Ketoprofen has not been expected to have any local side effects, and no side effects occurred from the treatment with our injections.

The pharmaceutical used by us affects the symptoms of the disease rapidly (approximately within 24 hours), the effect is long-acting or permanent. Compared to the orally administered antiinflammatory analgesics, the rapidity, the efficiency and the duration of the effect of the drug used by us are essentially better.

In the following we have used ketoprofen and diclofenac as the antiinflammatory analgesic. As the active agent of the glucocorticoid preparation we have used betamethasone, methylprednisolone and triamcinolone.

### PATIENT EXAMPLES

The patients treated by us included, among others, sixteen patients with chronic tennis elbow, whose disease had lasted from two months to one-year. All treated patients have recovered completely or nearly completely after two combination preparation injections (injected at one-week interval) within two to three weeks. The effect has lasted at least for three months from the first injection given.

In the following some additional examples of the patients:

| | | disease, | | treatment result | treatment result |
|---|---|---|---|---|---|
| patients | | duration | medical treatment* | after 1 week | after 2 weeks |
| 1. | 49 y. M | painful | ketoprofen + | slight pain and | no symptoms |
| | | tennis | betamethasone | minor clinical | no clinical findings |
| | | elbow, | injections | findings | |
| | | 6 months | | | |
| 2. | 43 y. M | painful | ketoprofen + | no visit at the | no symptoms, |
| | | tennis | betamethasone | doctor and | no clinical findings |
| | | elbow, | injections | no therapy | |
| | | 3 months | | | |
| 3. | 46 y. F | painful | ketoprofen + | slight pain and | no symptoms, |
| | | tennis | betamethasone | minor clinical | no clinical |
| | | elbow, | injections | findings | findings |
| | | 8 months | | | |
| 4. | 38 y. M | tendinitis | ketoprofen + | slight pain, | no symptoms |
| | | of the | betamethasone | minor clinical | |
| | | shoulder, | injections | findings | |
| | | 9 months | | | |
| 5. | 40 y. F | bursitis | ketoprofen + | slight pain and | no symptoms, |
| | | | betamethasone | minor clinical | no clinical |
| | | | injections | findings | findings |
| 6. | 49 y. F | bursitis | diclofenac + | slight pain and | nearly symptomless |
| | | | betamethasone | minor clinical | minor clinical |
| | | | injections | findings | findings |

*Two combinations preparation injections (injected at an interval of one week).

What is claimed is:

1. Pre-pack for the intralesional treatment of epicondylitis, tendinitis of the shoulder and bursitis containing a first component comprising at least one glukocorticoid and a second component comprising at least one non-steroidal antiinflammatory analgesic selected from the group consisting of phenyl acetic acid and phenyl propionic acid derivatives, and a pharmacologically acceptable carrier suitable for injection purposes and optionally further adjuvants and/or additives.

2. The pre-pack according to claim 1, wherein the pre-pack further contains means for combining the glucocorticoid in the first component, and the non-steroidal antiinflammatory analgesic in the second component to an injecting preparation containing the pharmacologically acceptable carrier suitable for injection purposes and the optional further adjuvants and/or additives.

3. The pre-pack according to claim 1, wherein the first component and the second component both comprise a preparation suitable for injection, each containing a pharmacologically acceptable carrier suitable for injection purposes and optionally further adjuvants and/or additives.

4. The pre-pack according to claim 2, wherein the first component and the second component both comprise a preparation suitable for injection, each containing a pharmacologically acceptable carrier suitable for injection purposes and optionally further adjuvants and/or additives.

5. The pre-pack according to claim 1, wherein the glucocorticoid is selected from the group consisting of cortisone, hydrocortisone, prednisone, prednisolone, betamethasone, dexamethasone, methylprednisolone, and triamcinolone.

6. The pre-pack according to claim 1, wherein the phenyl acid derivative is ketoprofen or diclofenac.

7. The pre-pack according to claim 1, wherein the carrier is water or an aqueous organic solvent.

8. The pre-pack according to claim 1, further containing a component comprising an anesthetic and, when necessary, adrenaline.
